(19) United States
(12) Patent Application Publication
(10) Pub. No.: US 2008/0146937 A1
(43) Pub. Date: Jun. 19, 2008

(54) MECHANICALLY EXPANDING TRANSDUCER ASSEMBLY

(75) Inventors: WARREN LEE, NISKAYUNA, NY (US); DOUGLAS GLENN WILDES, BALLSTON LAKE, NY (US); KAI ERIK THOMENIUS, CLIFTON PARK, NY (US); MIRSAID SEYED-BOLORFOROSH, GUILDERLAND, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 11/610,616

(22) Filed: Dec. 14, 2006

(51) Int. Cl. A61B 8/00

(52) U.S. Cl. 600/462

(57) ABSTRACT

A transducer assembly is presented. The transducer assembly includes a support structure configured to be reversibly changed between a first position and a second position. Additionally, the transducer assembly includes a multi-dimensional transducer array comprising a plurality ‘N’ of one-dimensional sub-groups of transducer elements arranged on the support structure, wherein each of the ‘N’ sub-groups of transducer elements is disposed in a spatial relationship such that an angle formed between one of the ‘N’ sub-groups of transducer elements and at least one other sub-group of transducer elements is less than about 180 degrees, and wherein ‘N’ is an integer.

### BACKGROUND

Embodiments of the invention relate generally to a transducer assembly, and more specifically to a transducer assembly for real-time imaging in space-constrained applications.

Transducers, such as acoustic transducers, have found application in medical imaging where an acoustic probe is held against a patient and the probe transmits and receives ultrasound waves. The received energy may, in turn, facilitate the imaging of the internal tissues of the patient. For example, transducers may be employed to image the heart of a patient.

A typical invasive probe may include a miniaturized transducer assembly disposed at a distal end of the probe. The probe may include, for example, a one-dimensional phased array transducer. As will be appreciated, spatial resolution of the transducer assembly is an important factor in imaging applications, such as ultrasound imaging. Additionally, acquisition of high quality real-time three-dimensional imaging volumes in space-constrained applications is disadvantageously dependent upon the number of signal conductors that may be accommodated within the limited space of the probe. Also, the relatively small physical size of a transducer assembly sized and configured for space-constrained applications unfortunately limits the aperture of the transducer assembly. This results in the generation of ultrasound beams that diverge rapidly with distance, thereby resulting in poor spatial resolution and degraded image quality. Consequently, the ability of a clinician to identify anatomical and physiological regions of interest may be hampered.

Currently available imaging catheters, such as ultrasound imaging catheters, typically have a side-viewing orientation where the ultrasound beam direction is generally perpendicular to a long axis of the imaging catheter. Although forward-looking catheters have emerged, the apertures are small and fixed resulting in poor resolution and penetration. Also, previously conceived solutions have incorporated one-dimensional catheter transducers to obtain three-dimensional images by rotating the entire catheter. However, the resulting images are not obtained in real-time.

Furthermore, previously conceived solutions for real-time three-dimensional imaging employ two-dimensional arrays to steer and focus the ultrasound beam over a pyramidal-shaped volume. However, many of these two-dimensional arrays require a relatively large number of interconnections in order to adequately sample the acoustic aperture space, resulting in increased cost and complexity.

### BRIEF DESCRIPTION

Briefly, in accordance with aspects of the invention, a transducer assembly is presented. The transducer assembly includes a support structure configured to be reversibly changed between a first position and a second position. Additionally, the transducer assembly includes a multi-dimensional transducer array comprising a plurality ‘N’ of one-dimensional sub-groups of transducer elements arranged on the support structure, wherein each of the ‘N’ sub-groups of transducer elements is disposed in a spatial relationship such that an angle formed between one of the ‘N’ sub-groups of transducer elements and at least one other sub-group of transducer elements is less than about 180 degrees, and wherein ‘N’ is an integer.

In accordance with further aspects of the technique, an invasive probe configured to image an anatomical region is presented. The invasive probe includes an outer envelope sized and configured to be disposed in the anatomical region. Further, the invasive probe includes a transducer assembly movably disposed in the outer envelope, where the transducer assembly includes a support structure configured to be reversibly changed between a first position and a second position, where the support structure includes a central guide member having a proximal end and a distal end, a plurality of support struts movably coupled to the distal end of the central guide member, and a sliding member coupled to the central guide member and the plurality of support struts to facilitate changing the support structure between the first position and the second position. The transducer assembly also includes a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.

In accordance with yet another aspect of the present technique, a system is presented. The system includes an acquisition subsystem configured to acquire image data, where the acquisition subsystem comprises an invasive probe configured to image an anatomical region, where the invasive probe includes an outer envelope sized and configured to be disposed in the anatomical region, and a transducer assembly movably disposed in the outer envelope. Furthermore, the transducer assembly includes a support structure configured to be reversibly changed between a first position and a second position and a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer. The support structure in the transducer assembly includes a central guide member having a proximal end and a distal end, a plurality of support struts coupled to the distal end of the central guide member and a sliding member movably coupled to the central guide member and the plurality of support struts to facilitate changing the support structure between the first position and the second position. Additionally, the system includes a processing subsystem in operative association with the acquisition subsystem and configured to process the image data acquired via the acquisition subsystem.

In accordance with further aspects of the present technique, a method of using an invasive probe having a transducer assembly is presented. The method includes positioning the invasive probe proximate a region of interest within an anatomical region, wherein the transducer assembly is in a first position and disposed in an outer envelope of the invasive probe. The method also includes extending the transducer assembly from within the outer envelope such that the transducer assembly is positioned outside a distal end of the invasive probe. Further, the method includes deploying the transducer assembly to change the position of the transducer assembly from the first position to a second expanded position, where the transducer assembly includes a support structure configured to be reversibly changed between a first position and a second position and a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.

In accordance with yet another aspect of the present technique, a method of using an invasive probe having a transducer assembly is presented. The method includes positioning the invasive probe proximate a region of interest within an anatomical region, where the transducer assembly is in a first position and disposed in an outer envelope of the invasive probe. Further, the method includes extending the transducer assembly from within the outer envelope such that the transducer assembly is positioned outside a distal end of the invasive probe. Additionally, the method includes imaging in a first retracted position. The method also includes transitioning the position of the transducer assembly from the first retracted position to a second expanded position to create an expanded acoustic aperture. Moreover, the method includes imaging in the second expanded position, where the transducer assembly includes a support structure configured to be reversibly changed between a first position and a second position and a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.

### DRAWINGS

These and other features, aspects, and advantages of the invention will become better understood when the following detailed description is read with reference to the accompanying drawings, which are provided for illustrative purposes and in which like characters represent like parts throughout the drawings, wherein:

FIG. 1 is a block diagram of an exemplary ultrasound imaging and therapy system, in accordance with aspects of the present technique;

FIG. 2 is a schematic diagram depicting an exemplary method for deploying an invasive probe for imaging in accordance with one embodiment of an exemplary mechanically expanding transducer assembly;

FIG. 3 is a schematic diagram depicting an exemplary method for deploying an invasive probe for imaging employing an invasive probe in accordance with an alternative embodiment of an exemplary mechanically expanding transducer assembly;

FIG. 4 is an end view of the exemplary embodiment of the mechanically expanding transducer assembly illustrated in FIGS. 2-3;

FIG. 5 is an end view of another embodiment of a mechanically expanding transducer assembly;

FIG. 6 is an end view of yet another embodiment of a mechanically expanding transducer assembly;

FIG. 7 is a perspective view of one exemplary embodiment of an invasive probe with a forward viewing three-dimensional volume orientation;

FIG. 8 is a perspective view of one embodiment of an invasive probe with a forward viewing three-dimensional volume orientation and configured to deliver therapy;

FIG. 9 is an end view of the embodiment of the invasive probe illustrated in FIG. 8; and

FIG. 10 is a perspective view of the embodiment of the invasive probe illustrated in FIG. 8.

### DETAILED DESCRIPTION

As will be described below, embodiments of the present technique include a transducer assembly having a support structure and a multi-dimensional transducer array configured such that the transducer array may be reversibly transitioned from first radially collapsed position to a second radially expanded position.

Although the exemplary embodiments illustrated hereinafter are described in the context of a medical imaging system, such as an ultrasound imaging system, other imaging systems and applications such as industrial imaging systems and non-destructive evaluation and inspection systems, such as pipeline inspection systems, liquid reactor inspection systems are also contemplated. Additionally, the exemplary embodiments illustrated and described hereinafter may find application in multi-modality imaging systems that employ an ultrasound imaging in conjunction with other imaging modalities, position-tracking systems or other sensor systems.

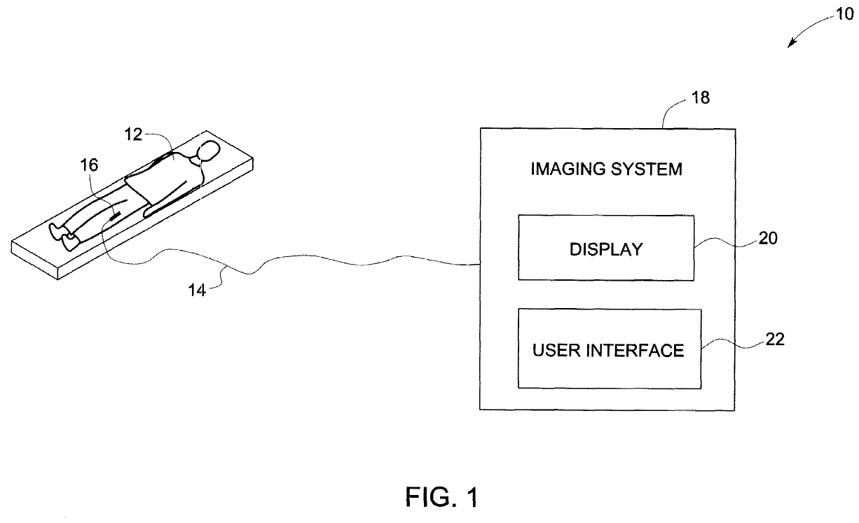

FIG. 1 is a block diagram of an exemplary system 10 for use in imaging, in accordance with aspects of the present technique. The system 10 may be configured to acquire image data representative of a region of interest in a patient 12 via a probe 14. As used herein, the term “probe” is broadly used to include conventional catheters, transducers or devices adapted for imaging and applying therapy. Further, as used herein, the term “imaging” is broadly used to include two-dimensional (2D) imaging, three-dimensional (3D) imaging, or real-time three-dimensional (RT3D) imaging. It may be noted that the terms RT3D and four-dimensional (4D) imaging may be used interchangeably.

In accordance with aspects of the present technique, the probe 14 may be configured to facilitate interventional procedures in which the probe 14 may be configured to function as an invasive probe. It should also be noted that, although the illustrated embodiments are described in the context of a catheter-based probe, other types of probes such as endoscopes, laparoscopes, surgical probes, transrectal probes, transvaginal probes, intracavity probes, probes adapted for interventional procedures, or combinations thereof are also contemplated in conjunction with the present technique. Reference numeral 16 is representative of a portion of the probe 14 disposed inside the patient 12.

Further, in the illustrated embodiment, an imaging system 18 is in operative association with the invasive probe 14. The imaging system 18 may be configured to display an image representative of a current position of the invasive probe 14 within a region of interest in the patient 12. The imaging system 18 may include a display 20 and a user interface 22. In accordance with aspects of the present technique, the display 20 of the imaging system 18 may be configured to display images generated by the imaging system 18 based on image data acquired via the invasive probe 14.

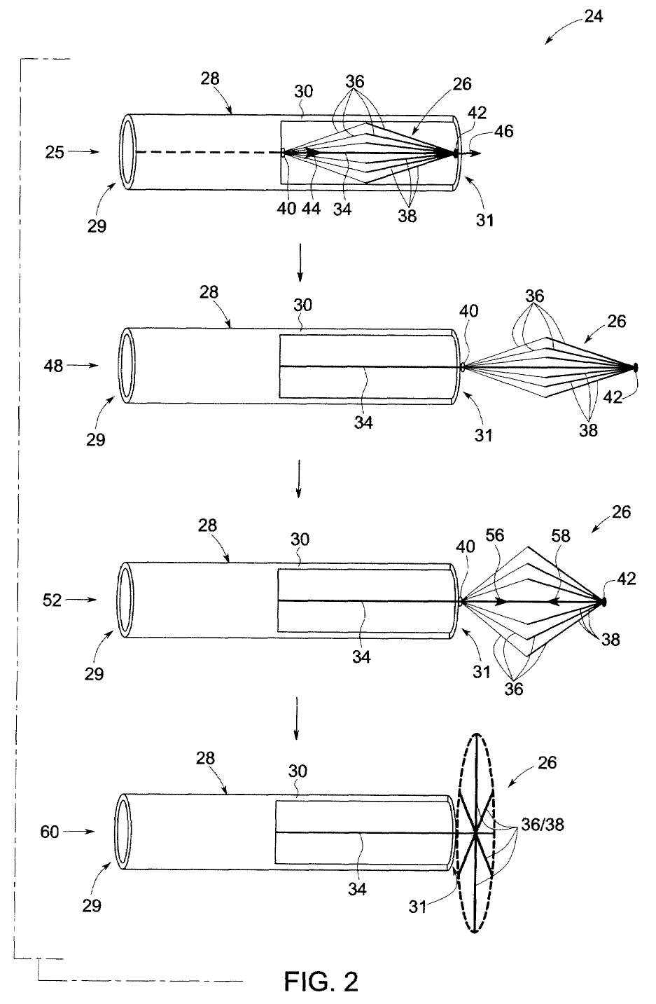

Turning now to FIG. 2, a schematic diagram 24 depicting an exemplary method for deploying an invasive probe 28 including a mechanically expanding transducer assembly 26 is illustrated in accordance with one embodiment. Reference numeral 25 is representative of the invasive probe 28 illustrating (in a cut-away view) the transducer assembly 26 situated in a first radially collapsed position.

As illustrated, the invasive probe 28 generally includes a proximal end 29 and a distal end 31, and is shown as including an outer envelope 30. In one embodiment, the transducer assembly 26 is illustrated as being disposed within the outer envelope 30 of the invasive probe 28. Alternatively, in another embodiment, the transducer assembly 26 may be disposed at an end of the outer envelope 30 of the transducer assembly. In one embodiment, the transducer assembly 26 may be disposed at the distal end 31 of the outer envelope 30 of the invasive probe 28, for example. In accordance with aspects of the present technique, the transducer assembly 26 may include a support structure configured to be reversibly changed between at least a first position and a second position. Further, for the illustrated embodiment, the first position may include a radially collapsed position, while the second position may include a radially expanded position. Accordingly, the size of the expanded transducer array need not be limited by the catheter diameter. Moreover, in one embodiment, the size of the transducer array in the expanded position may be larger than catheter diameter as measured in at least two dimensions. In an embodiment where the expanded transducer array may be represented with a diameter, the diameter of the expanded transducer array may be larger than the catheter diameter as measured in a direction orthogonal to the axis of the catheter. In the first radially collapsed position, the transducer assembly 26 may be configured in a compact folded state having a form factor designed to fit within the outer envelope 30 for delivery to a region of interest. In one embodiment, the transducer assembly 26 may be configured to facilitate imaging in the radially collapsed position, the radially expanded position, or both.

The support structure may include a central guide member 34 that extends through the center of the transducer assembly 26 and is defined to include a proximal end 29 and a distal end 31 corresponding to that of the invasive probe 28. In one embodiment, the central guide member 34 may constructed from metals suitable for medical devices, such as stainless steel, nitinol, titanium, etc. Also, the central guide member 34 may include any of a variety of cross-sections including a circular cross-section, in certain embodiments. In such an embodiment, the central guide member 34 may have a diameter in a range from about 0.1 mm to 2 mm.

In one embodiment, a first end (for example, a distal end) of the support structure may be movably coupled to the central guide at or about the distal end 31 of the invasive probe 28 and a second end (for example, a proximal end) of the support structure may be movably coupled to an intermediate portion of the central guide member 34. In one embodiment the support structure includes multiple radial struts 36 coupled between the two ends of the support structure. The support structure may also include a sliding member 40 such as a slip ring and a hinge connection 42 coupled to the central guide member 34 as shown to facilitate transitioning the support structure between the collapsed and expanded positions. Reference numeral 46 is representative of a first direction of movement of the transducer assembly 26 whereas a subsequent direction of movement of the sliding member 40 along the central guide member 34 is indicated by reference numeral 44. In one embodiment, the radial struts 36 are movably coupled to the central guide member 34 via the sliding member 40 and hinge connection 42. Also, in accordance with one embodiment, at least one of the plurality of radial struts 36 may include a flexible circuit. The flexible circuit may include single or multi-layer copper circuit(s) on a polyimide substrate, in certain embodiments.

In certain embodiments, two or more transducer elements (not shown) may be arranged on the support structure to facilitate imaging of a region of interest. In one embodiment, one or more transducer elements may be disposed on each of the radial struts 36. Reference numeral 38 is representative of radial struts having two or more transducer elements disposed thereon. Furthermore, the transducer elements may be arranged on the radial struts in a pseudo-random pattern, a vernier pattern or other patterns to facilitate minimizing grating lobes and other beamforming artifacts. The transducer elements may include lead zirconate titanate (PZT) transducer elements, capacitively micromachined ultrasound transducer (cMUT) elements or polyvinylidene fluoride array (PVDF) transducer elements.

In a presently contemplated configuration, the transducer elements may be arranged on the radial struts 38 to form sub-groups of transducer elements. In one embodiment, the radial struts 38 may contain a number “N’ (where N is an integer value) of sub-groups of transducer elements. In one embodiment, a number ‘N’ of sub-groups of transducer elements may be arranged on the support structure such that an angle formed between one of the sub-groups of transducer elements on one radial strut and at least one other sub-group of transducer elements on another radial strut is less than about 180 degrees. In one embodiment, the angles of separation between each neighboring radial strut of the transducer assembly 26 may be substantially equivalent and may be determined according to the following relationship:

$$Separationangle = ( \frac{2 \times 180}{Numberofradialstruts} ) \qquad ( 1 )$$

where the separation angle may be measured in degrees.

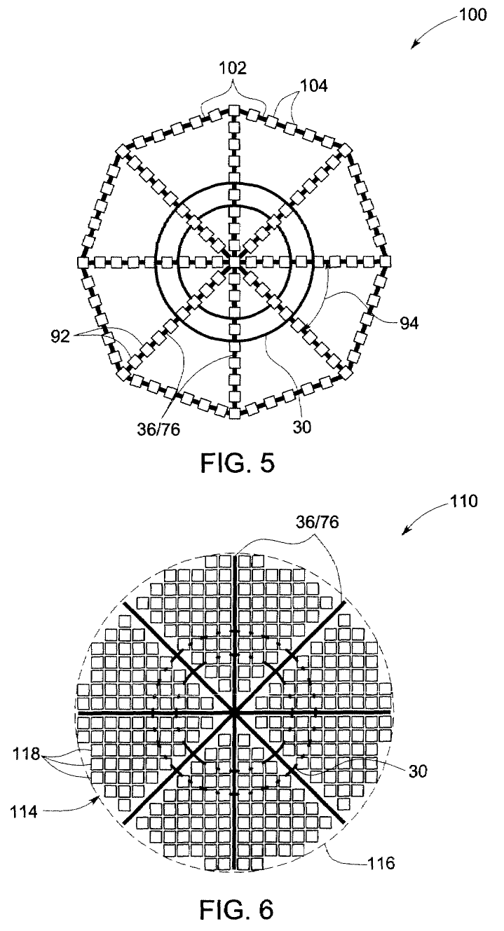

For example, in one embodiment the measure of an angle between each neighboring radial strut in a transducer assembly having 4 radial struts may be equal to about 90 degrees. In one embodiment, the support structure may also include spacers (not shown) coupled to the radial struts 36. As will be discussed in further detail below with reference to FIG. 5, the spacers may be configured to control spacing between the radial struts 36 in the expanded position. It may be noted that in certain embodiments, two or more sub-groups of transducer elements also may be arranged on each of the radial struts 38 or a subset of the radial struts 38. Additionally, the sub-groups of transducer elements may be referred collectively to as a multi-dimensional transducer array.

According to further aspects of the present technique, the invasive probe 28 having a mechanically expanding transducer assembly, such as the transducer assembly 26, may be employed to facilitate imaging in space-constrained applications, such as, but not limited to, intracardiac echocardiography, transesophageal echocardiography, pediatric echocardiography, laparoscopic surgery. More particularly, the invasive probe 28 equipped with the transducer assembly 26 may be employed to obtain high quality RT3D image volumes.

The method of imaging employing the invasive probe 28 having the exemplary transducer assembly 26 may include positioning the invasive probe 28 proximate a region of interest within an anatomical region in the patient 12. The invasive probe 28 may be guided from the point of entry through the vasculature of the patient 12 to the desirable anatomical location employing a method such as fluoroscopy to monitor and guide the invasive probe 28 within the vasculature. The invasive probe could be delivered over a guide wire or through a sheath, where the sheath or wire had previously been guided to the desired location using e.g., fluoroscopic imaging. Once delivered to the region to be imaged, the transducer elements in the multi-dimensional transducer array may be energized and image data representative of the region of interest may be acquired. Image data may be acquired via the transducer assembly 26 while positioned within the outer envelope 30 of the invasive probe 28 (e.g., in a radially collapsed configuration) or while positioned outside of the outer envelope 30 (e.g., in the radially collapsed configuration or an expanded configuration). It may be noted that by employing the transducer assembly 26 in the radially collapsed position, multiple image planes may be acquired. For example, each “arm” of the transducer assembly 26 may be configured to operate independently to acquire a separate image plane.

As alluded to above, the transducer assembly 26 may be moved from within the outer envelope 30 such that the transducer assembly 26 is positioned outside the distal end 31 of the invasive probe 28 in order to image a region of interest. Reference numeral 48 is representative of the invasive probe 28 in which the transducer assembly 26 is positioned outside the distal end 31 of the invasive probe 28. In one embodiment, the transducer assembly 26 and the central guide member 34 may be extended out of the distal end 31.

Once the transducer assembly 26 has been positioned outside the distal end 31 of the invasive probe 28, the transducer assembly 26 may be transitioned from the first radially collapsed position to a second radially expanded position, in which the aperture of the transducer assembly 26 is not limited by the diameter of the catheter. Moreover, in one embodiment, the size of the transducer array in the expanded position may be larger than catheter diameter as measured in at least two dimensions. In an embodiment where the expanded transducer array may be represented with a diameter, the diameter of the expanded transducer array may be larger than the catheter diameter as measured in a direction orthogonal to the axis of the catheter. For example, the diameter of an intracardial catheter may be in the range of about 1 mm to 4 mm, while the aperture of the transducer assembly 26 in the second expanded position may be in a range from about 3 mm to 30 mm.

Reference numeral 52 is representative of the invasive probe 28 in which the transducer assembly 26 is depicted in an intermediate or partially deployed position that is between the radially collapsed position and the radially expanded position. Image data representative of the region of interest may also be acquired via the transducer assembly 26 in the partially deployed position. It may be noted that the transducer assembly 26 in the partially deployed position may be employed to obtain multiple image planes. For example, the arms of the transducer assembly 26 may be phased together depending upon the position of the arms during the intermediate position of deployment. Alternatively, the arms may act independently to acquire multiple image planes. In one embodiment, imaging of the entire forward hemisphere may be accomplished while the arms of the transducer assembly 26 are positioned at about 45 degrees as measured with respect to the catheter axis.

Deployment of the transducer assembly 26 to the radially expanded position may be achieved via the use of mechanical wires, in certain embodiments. Alternatively, shape memory material may be utilized to facilitate the deployment of the transducer assembly 26. Furthermore, hinges made using electro-actuated polymer actuators may also be employed to aid in transitioning the transducer assembly 26 from the radially collapsed position to the radially expanded position. In certain other embodiments, the transducer assembly 26 may be transitioned from the radially collapsed position to the intermediate or partially deployed position by retracting the hinge connection 42 with respect to the radial struts 36 in a direction represented by reference numeral 58 In other embodiments, the transducer assembly 26 may be transitioned to the intermediate or partially deployed position by extending the slip ring 40 over the central guide member 34 in a direction indicated by reference numeral 56, while maintaining the hinge connection 42 in a fixed location.

Reference numeral 60 is representative of the invasive probe 28 having the transducer assembly 26 in a full radially expanded position. As previously noted, the acoustic aperture of the transducer assembly 26 in the radially expanded position may be in a range from about 3 mm to 30 mm, however larger acoustic apertures are possible. It may be noted that the transducer assembly 26 in the radially expanded position may be configured to have a forward viewing orientation. Additionally, the transducer assembly 26 in the radially expanded position may be used to acquire image data representative of the region of interest. More particularly, image data may be acquired employing the transducer assembly 26 in the radially expanded position.

Once the image data is acquired via the transducer assembly 26 in the expanded configuration of 60, the transducer assembly 26 may be transitioned back to the radially collapsed position. The transducer assembly 26 may be transitioned to the radially collapsed position from the radially expanded position by moving the sliding member 40 in a second direction (opposite to direction 44) along the central guide member 34. In certain other embodiments, the transducer assembly 26 may be transitioned to a radially collapsed position through use of a pull wire (not shown) or active hinges (not shown).

The transducer assembly 26 in the radially collapsed position subsequently may be retracted such that the transducer assembly 26 is once again positioned within the outer envelope 30 of the invasive probe 28. The invasive probe 28 along with the transducer assembly 26 in the radially collapsed position may then be removed from the anatomy of the patient by a clinician, for example.

As will be appreciated, the respective positions of the plurality of transducer elements may experience a positional shift while the transducer assembly 26 is transitioned between the radially collapsed position and the radially expanded position. As such, it is desirable to determine precise locations (e.g., to within a fraction of a wavelength to allow for proper phasing) of the plurality of transducer elements in the transducer assembly 26 to facilitate generation of a high-quality image using image data acquired via the plurality of transducer elements. In certain embodiments, adaptive beamforming techniques may be used to compensate for the variations and/or positional shifts of the plurality of transducer elements. The acquired image data may then be utilized to generate an image for display on the display 20 (see FIG. 1) of the imaging system 18 (see FIG. 1), for example.

By implementing the transducer assembly 26 as described hereinabove, RT3D image volumes having relatively enhanced quality may be obtained employing the transducer assembly 26 in the radially expanded position. Additionally, the transducer assembly 26 described hereinabove may be configured to have an alternative compact structure such that the transducer assembly 26 in the radially collapsed position may be configured to fit within a space-constrained invasive probe 28. Consequently, high quality RT3D image volumes may be acquired using the large aperture transducer assembly 26 that may also be inserted and removed using a narrow probe configured for minimally invasive applications, such as, but not limited to, intracardiac echocardiography, transesophageal echocardiography, pediatric echocardiography, laparoscopic surgery.

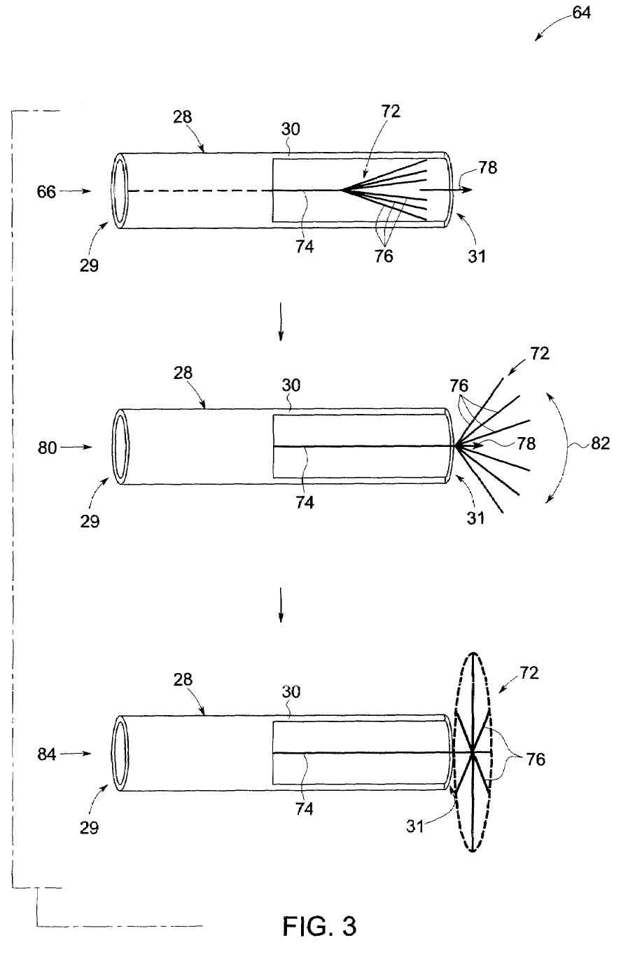

Turning now to FIG. 3, a schematic diagram 64 depicting an exemplary method for deploying an invasive probe 28 including an alternative embodiment of a mechanically expanding transducer assembly is illustrated. Reference numeral 66 is representative of the invasive probe 28 including a mechanically expanding transducer assembly 72 in a first radially collapsed position.

As previously described with respect to the transducer assembly 26 of FIG. 2, the transducer assembly 72 may also include a support structure upon which the transducer assembly 72 is supported. Such a support structure may include a central guide member 74 having a first (proximal) end 29 and a second (distal) end 31 and may be configured to facilitate transitioning the transducer assembly 72 from a first position to a second position. The first position may include a radially collapsed position, while the second position may include a radially expanded position, where the acoustic aperture of the transducer assembly 72 in the radially expanded position is not limited by the diameter of the catheter as previously described.

Additionally, the transducer assembly 72 may include a plurality of support struts 76, in which the plurality of support struts has a respective proximal end and a distal end. In a presently contemplated configuration, the respective proximal ends of the plurality of support struts 76 may be coupled to the central guide member 74 at the distal end of the central guide member 74 as shown in FIG. 3. The plurality of support struts 76 may be formed from a wire in certain embodiments. More particularly, the support struts 76 may be formed from or otherwise include a shape-memory wire or a spring wire, for instance. The shape-memory wire may include Nitinol, in certain embodiments. The shape-memory wire or the spring wire may be configured to automatically transition the transducer assembly 72 from the radially collapsed position to the radially expanded position when extended from within the outer envelope 30 of the invasive probe 28 in a direction of movement generally represented by reference numeral 78.

Furthermore, a plurality of transducer elements (not shown) may be disposed on the plurality of support struts 76. The transducer elements may be physically or electrically configured into sub groups of transducer elements that together form a multi-dimensional transducer array. In one embodiment, the support struts 76 may contain a number ‘N’ (where N is an integer value) of sub-groups of transducer elements. In one embodiment, a number ‘N’ of sub-groups of transducer elements may be arranged on the support structure such that an angle formed between one of the sub-groups of transducer elements on one support strut and at least one other sub-group of transducer elements on another support strut is less than about 180 degrees.

A method of imaging employing the invasive probe 28 having the exemplary transducer assembly 72 may include positioning the invasive probe 28 at a region of interest within an anatomical region, as previously described with reference to FIG. 2. Subsequently, as illustrated by reference numeral 80, the transducer assembly 72 may be extended from within the outer envelope 30 such that the transducer assembly 72 is positioned outside the distal end 31 of the invasive probe 28. In one embodiment, the outer envelope 30 acts to maintain the transducer assembly in the radially collapsed configuration. As the transducer assembly 72 is extended out of the outer envelope 30, the support struts 76 are free to spread apart into a radially expanded configuration. Reference numeral 80 is representative of the invasive probe 28 where the transducer assembly 72 has been extended from within the outer envelope 30 and is configured in a partially deployed position. Further, a direction of expansion of the support struts 76 may be generally represented by reference numeral 82.

Reference numeral 84 is indicative of the invasive probe 28 where the transducer assembly 72 is configured in the radially expanded position. As previously noted, the transducer assembly 72 may be transitioned to the radially expanded position to create an acoustic aperture that is larger than the diameter of the catheter. Image data representative of the region of interest may be acquired via the transducer assembly 72 in the radially expanded position. Furthermore, as previously described with reference to FIG. 2, image data representative of the region of interest may also be obtained via the transducer assembly 72 in the radially collapsed position, the second radially expanded position or a position therebetween.

Following acquisition of image data using the transducer assembly 72 in the radially expanded position, the transducer assembly 72 may subsequently be retracted into the outer envelope 30 of the invasive probe 28. In one embodiment, retraction of the transducer assembly 72 in the radially expanded position into the outer envelope 30 of the invasive probe 28 urges the support struts 76 together causing the transducer assembly 72 to transition to the radially collapsed position.

Moreover, adaptive beamforming techniques may be employed to compensate for positional shifts and/or variations of the plurality of transducer elements, as previously described with reference to FIG. 2. An image representative of the region of interest may be subsequently generated using the acquired image data, where the image may then be displayed on a display, such as the display 20 (see FIG. 1) of an imaging system, such as imaging system 18 (see FIG. 1).

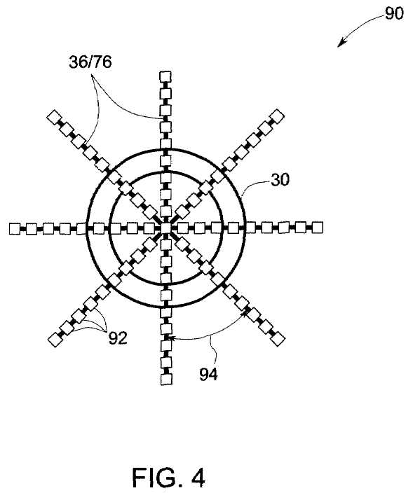

FIG. 4 is an end view 90 of the mechanically expanding transducer assembly such as transducer assembly 26 (see FIG. 2) or transducer assembly 72 (see FIG. 3) illustrated in FIGS. 2-3, where the transducer assembly 26/72 are depicted in a radially expanded position. As previously noted, the mechanically expanding transducer assembly includes a plurality of support struts, such as radial struts 36/76, for example. Furthermore, as previously noted a plurality of transducer elements 92 may be disposed on each of the radial struts 36/76 to form sub-groups of transducer elements. Additionally, as described hereinabove, the sub-groups of transducer elements may be arranged in a spatial relationship such that an angle formed between a first sub-group of transducer elements on a first radial strut 36/76 and a second sub-group of transducer elements on at least one other radial strut 36/76 is less than about 180 degrees. For example, reference numeral 94 is representative of an angle formed between a first sub-group of transducer elements and a second sub-group of transducer elements, where the angle 94 is less than about 180 degrees.

In the mechanically expanding transducer assembly depicted in FIG. 4, eight (8) imaging arms (corresponding to struts 76) are shown. In general, the more struts 36/76 (and associated transducer elements) that are utilized, the more filled-in (e.g., less sparse) the imaging aperture becomes, and the more the sidelobes and grating lobes can be suppressed. This in turn acts to improve image quality providing e.g., better contrast and fewer artifacts. In certain embodiments, the presence of grating lobes and side lobes may be reduced by using specific transducer element sub-groups on transmit and receive that place the grating lobes and side lobes at different angular positions so that their transmit-receive product is minimized.

FIG. 5 is an end view 100 of an alternative embodiment of a mechanically expanding transducer assembly such as transducer assembly 26 (see FIG. 2) or transducer assembly 72 (see FIG. 3). The transducer assembly is illustrated in a fully deployed, radially expanded position. In accordance with further aspects of the present technique, spacers may be coupled to the radial struts 36/76 to control spacing between the radial struts 36/76 in the radially expanded position. As shown in the illustrated embodiment of FIG. 5, spacers may include circumferential struts 102 disposed between some or all of the radial struts 36/76. In one embodiment the plurality of circumferential struts 102 may be coupled between the distal ends of each of the radial struts 36/76. Additionally, transducer elements 104 may be disposed on one or more of the circumferential struts 102 to form a respective circumferential sub-group of transducer elements. In accordance with further aspects of the present technique, spacers may also take the form of a web or a cord coupled between the radial struts 36/76. In one embodiment, a cord spacer may be thin and flexible, whereas a “strut” spacer may be relatively stiffer. Struts 102 may act to push the radial struts 36/76 apart and hold them at a fixed spacing. In one embodiment, cords may connect all radial struts (as shown in FIG. 5) and the radial struts or the central hinge would act to hold the radial struts open, placing the cord in tension. The cord's role is then to maintain uniform spacing, and perhaps to support transducer elements 104. The advantage of using a cord spacer is that, being thin and flexible, it would more easily fold up and fit into the catheter.

Advantages of using spacers, including circumferential struts, include more accurate and reproducible positioning of the radial struts to reduce phasing errors, or requirement to use adaptive imaging to compensate for positional variations. Additionally, if the spacers support transducer elements, they help fill-in the aperture and thereby improve the image quality (e.g., penetration & contrast).

FIG. 6 is an end view 110 of yet another embodiment of a mechanically expanding transducer assembly, such as transducer assembly 26 (see FIG. 2) or transducer assembly 72 (see FIG. 3), including a webbed transducer array 114. Such a webbed transducer array 114 may include a plurality of transducer elements 118 formed by metallizing and poling a piezoelectric polymer webbing, such as polyvinylidene fluoride (PVDF). Alternatively, a traditional PZT acoustic stack could be constructed on a flexible substrate (e.g. a polyimide flex circuit), and the stack could be diced to form a 2D array. The dicing depth should extend to or just into the polyimide, so the result is flexible. The dicing “streets” should be relatively wide, so the array can fold with elements face-to-face as well as back-to-back, to fit into the catheter. It may be noted that the flexible substrate 116 may have a first side and a second side and the transducer elements 118 may be disposed on the first side of the flexible substrate 116, the second side of the flexible substrate 116, or both. The configuration shown in FIG. 6 provides a more densely sampled 2D array. This increases the signal-to-noise ratio and thus image penetration, and also reduces grating lobes and side lobes, thus improving image contrast.

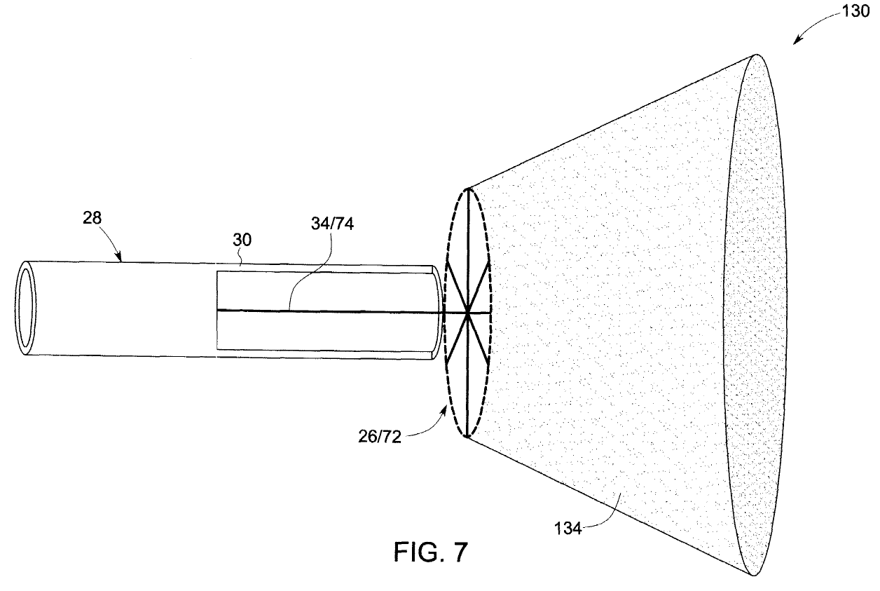

Referring now to FIG. 7, a perspective view 130 of one exemplary embodiment of an invasive probe 28 with a forward viewing three-dimensional volume orientation is illustrated. Depending on the application, the transducer may be used to image all or just a portion of the indicated 3D volume 134. For instance, imaging the full volume allows a full, rendered 3D view, but imaging only two or three slices within that volume (e.g., parallel to the spokes of the array) would allow a much faster image update rate. As previously noted, the invasive probe 28 is shown as including an outer envelope 30 and a transducer assembly such as the transducer assembly 26 (see FIG. 2) or the transducer assembly 72 (see FIG. 3). In the illustrated embodiment, the transducer assembly 26/72 is shown in a fully deployed position. Reference numeral 134 is representative of a three-dimensional forward viewing imaging volume of the invasive probe 28 having the exemplary mechanically expanding transducer assembly 26/72.

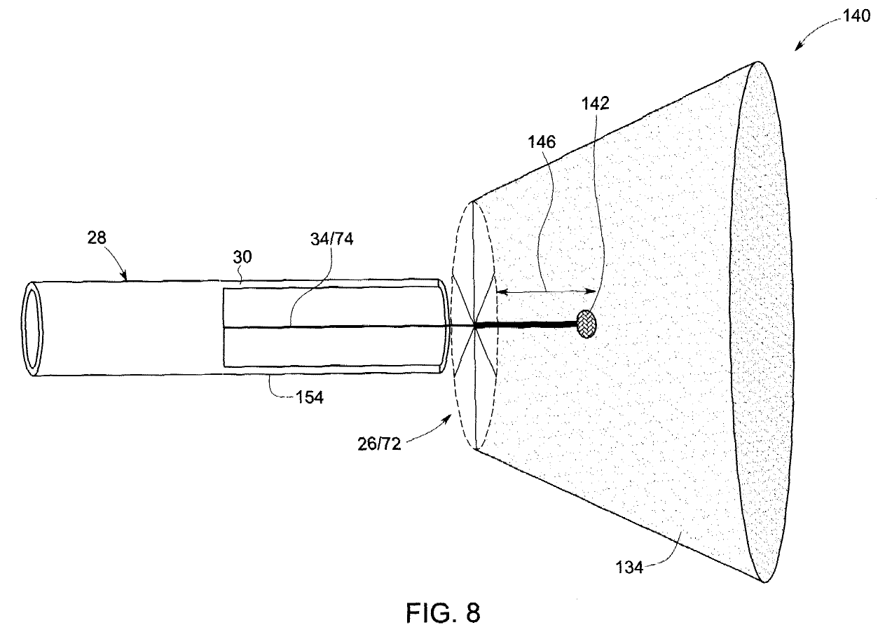

As described hereinabove, the invasive probe 28 equipped with the various configurations of the mechanically expanding transducer assembly such as the configurations illustrated in FIGS. 2-6, for example, may be employed to facilitate imaging of one or more regions of interest within the anatomy of the patient. According to aspects of the present technique, the invasive probe 28 equipped with one or more configurations of the mechanically expanding transducer assembly may also be configured to aid in delivery of therapy. FIG. 8 is a perspective view 140 of one embodiment of an invasive probe 28 having a forward viewing three-dimensional volume orientation and that is further configured to deliver therapy. It may be noted that the transducer assembly 26/72 (see FIGS. 2-3) is illustrated in a radially expanded position.

In addition to facilitating imaging of a region of interest, the invasive probe 28 having the mechanically expanding transducer assembly 26/72 may also be employed to facilitate delivery of therapy to one or more regions of interest in an anatomical region. As previously described, the invasive probe 28 may be positioned in the region of interest prior to imaging and/or delivery of therapy. In certain embodiments, placement of the invasive probe 28 within the anatomy may be performed under fluoroscopic guidance.

In accordance with aspects of the present technique, the invasive probe 28 may be configured to image an anatomical region to facilitate assessing need for therapy in one or more regions of interest within the anatomical region of the patient 12 (see FIG. 1) being imaged. Additionally, the invasive probe 28 may also be configured to deliver therapy to the identified one or more regions of interest. Accordingly, the invasive probe 28 may also include a therapy component 142 that may be configured to deliver therapy to the region of interest.

As used herein, “therapy” is representative of ablation, percutaneous ethanol injection (PEI), cryotherapy, and laser-induced thermotherapy. Additionally, “therapy” may also include delivery of tools, such as needles for delivering gene therapy, or biopsy forceps for example. Additionally, as used herein, “delivering” may include various means of providing therapy to the one or more regions of interest, such as conveying therapy to the one or more regions of interest or directing therapy towards the one or more regions of interest. As will be appreciated, in certain embodiments the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

In one embodiment, the imaging system, such as the imaging system 18 (see FIG. 1) may be configured to provide control signals to the invasive probe 28 to excite the therapy component 142 and deliver therapy to the one or more regions of interest. In one embodiment, the therapy component 142 of the invasive probe 28 may include an extendable and/or retractable device. More particularly, the therapy component 142 may include an electro-physiological mapping electrode, a monitoring or ablation electrode, a biopsy needle, a needle for trans-septal puncture, a fiber optic with a lens at the end, a tube with a sampling loop extending from the end, or combinations thereof. Although illustrated as such, the therapy device isn't required to extend from the very center of the transducer array, but could also be offset or extend from some of the spaces between the support struts. The direction of movement of the therapy component 142 is generally represented by reference numeral 146.

By implementing the invasive probe 28 having a mechanically expanding transducer assembly such as the transducer assembly 26 or the transducer assembly 72 and the therapy component 142 as described hereinabove, the therapy component 142 is generally in alignment with the imaged volume 134, thereby allowing clinicians to efficiently guide and place the therapy component 142 at a desired location with enhanced ease.

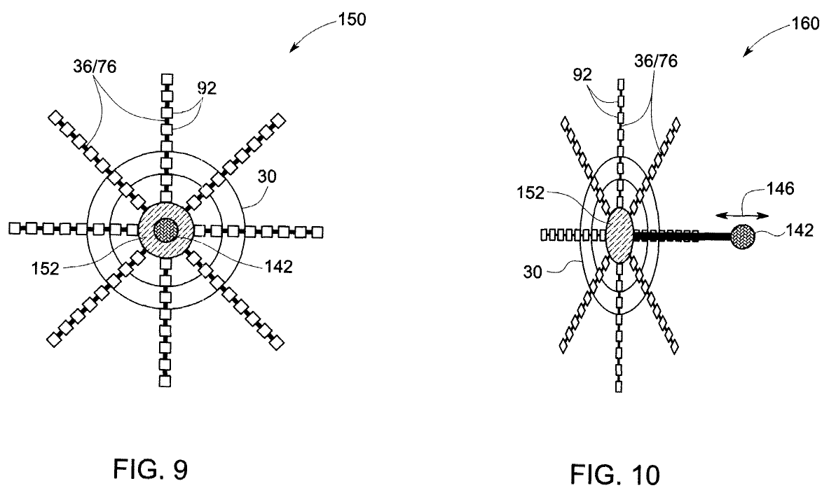

FIG. 9 is an end view 150 of the embodiment of the invasive probe assembly 28 illustrated in FIG. 8. As previously described, a plurality of transducer elements 92 may be disposed on the plurality of radial struts 36/76. Additionally, a working port 152 may be disposed within a lumen of the invasive probe 28 in addition to the exemplary mechanically expanding transducer assembly, as illustrated in FIG. 9. The working port 152 may be configured to facilitate deployment of the retractable therapy component 142. In one embodiment, the working port 152 may be configured to run the entire length of the invasive probe 28. Alternatively, the central guide member 34/74 (see FIG. 8) may be employed for integrating the therapy component 142 into the transducer assembly. Furthermore, the working port 152 may be configured to facilitate delivery of therapy to one or more regions of interest. If working port 152 is positioned off-center, between two of the arms 36/76, then rotating the entire catheter in-place would move the therapy port to different locations, allowing delivery of therapy to multiple regions of interest.

FIG. 10 illustrates a side view 160 of the embodiment of the invasive probe 28 illustrated in FIG. 8. In the illustrated embodiment, the therapy component 142 is shown in an extended position.

The various embodiments of the mechanically expanding transducer assemblies and the invasive probes having the mechanically expanding transducer assemblies configured to image and provide therapy and method of imaging and providing therapy described hereinabove dramatically enhance efficiency of the process of imaging and delivering therapy, by integrating the imaging and therapy mapping aspects of the procedure. Employing the transducer assembly described hereinabove allows acquisition of high quality RT3D images from within a space-constrained environment, such as the invasive probe. In addition, the unfolding of the transducer assemblies to obtain a relatively large acoustic aperture facilitates generation of images with relatively higher spatial resolution as compared to those images generated by transducer assemblies having relatively small acoustic apertures. Additionally, the forward viewing configuration of the transducer assembly integrated with diagnostic and/or therapeutic tools advantageously result in enhanced visualization of regions of interest, and simplified interventional procedures.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A transducer assembly, comprising:
a support structure configured to be reversibly changed between a first position and a second position; and
a multi-dimensional transducer array comprising a plurality ‘N’ of sub-groups of transducer elements arranged on the support structure, wherein each of the ‘N’ sub-groups of transducer elements is disposed in a spatial relationship such that an angle formed between one of the ‘N’ sub-groups of transducer elements and at least one other sub-group of transducer elements is less than about 180 degrees, and wherein ‘N’ is an integer.

2. The transducer assembly of claim 1, wherein the first position is a radially collapsed position and the second position is a radially expanded position.

3. The transducer assembly of claim 2, wherein a size of an aperture of the transducer assembly in the second position, as measured in a direction orthogonal to a long axis of the catheter, is larger than the diameter of the catheter.

4. The transducer assembly of claim 1, wherein the multi-dimensional transducer array is configured to have a forward viewing orientation in the second position.

5. The transducer assembly of claim 1, wherein the support structure comprises:
a central guide member having a proximal end and a distal end; and
a plurality of radial struts movably coupled to the distal end of the central guide member to provide support to the multi-dimensional transducer array.

6. The transducer assembly of claim 5, further comprising a sliding member coupled to the central guide member and the plurality of radial struts to facilitate changing the support structure between the first position and the second position.

7. The transducer assembly of claim 5, wherein at least one of the radial struts comprises a flexible circuit.

8. The transducer assembly of claim 5, further comprising at least one spacer coupled to at least two of the plurality of the radial struts and configured to control spacing between the at least two radial struts in the second position.

9. The transducer assembly of claim 5, wherein the plurality of radial struts comprises transducer elements arranged thereon.

10. The transducer assembly of claim 5, further comprising a web and a plurality of transducer elements disposed thereon.

11. The transducer assembly of claim 10, further comprising a plurality of circumferential struts coupled between distal ends of each of the plurality of the radial struts.

12. The transducer assembly of claim 11, wherein a plurality of transducer elements is arranged on the plurality of circumferential struts.

13. The transducer assembly of claim 7, wherein the flexible circuit comprises a flexible substrate having a first side and a second side and disposed on the plurality of the radial struts.

14. The transducer assembly of claim 13, wherein the plurality of transducer elements is arranged on a first side of the flexible substrate such that the plurality of transducer elements is disposed between the plurality of the radial struts.

15. The transducer assembly of claim 1, wherein the transducer array comprises a lead zirconate titanate array, a micromachined ultrasound array, a polyvinylidene fluoride array, or a combination thereof.

16. An invasive probe configured to image an anatomical region, comprising:
an outer envelope sized and configured to be disposed in the anatomical region; and
a transducer assembly disposed in or on the outer envelope, the transducer assembly comprising
a support structure configured to be reversibly changed between a first position and a second position, wherein the support structure comprises
a central guide member having a proximal end and a distal end;
a plurality of support struts movably coupled to the the central guide member near the distal end;
wherein the central guide member moves relative to the outer envelope to facilitate changing the support structure between the first position and the second position; and
a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.

17. The invasive probe of claim 16, wherein the invasive probe comprises an imaging catheter, an endoscope, a laparoscope, a surgical probe, a transesophageal probe, a transvaginal probe, a transrectal probe, an intracavity probe, or a probe adapted for interventional procedures.

18. The invasive probe of claim 16, wherein the first position is a radially collapsed position and the second position is a radially expanded position.

19. The invasive probe of claim 16, wherein the plurality of transducer elements is arranged on the plurality of support struts.

20. The invasive probe of claim 16, wherein the invasive probe is further configured to facilitate assessing the need for therapy in one or more regions of interest within the anatomical region and delivering therapy to the one or more regions of interest within the anatomical region.

21. A system, comprising:
an acquisition subsystem configured to acquire image data, wherein the acquisition subsystem comprises an invasive probe configured to image an anatomical region, and a processing subsystem in operative association with the acquisition subsystem and configured to process the image data acquired via the acquisition subsystem;
wherein the invasive probe comprises an outer envelope sized and configured to be disposed in the anatomical region, and a transducer assembly movably disposed in or on the outer envelope, the transducer assembly comprising
a support structure comprising a central guide member having a proximal end and a distal end, and a plurality of support struts coupled to the central guide member, wherein the central guide member moves relative to the outer envelope to facilitate changing the support structure between a first position and a second position; and
a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.

22. The system of claim 21, wherein the processing subsystem comprises an imaging system, wherein the imaging system comprises an ultrasound imaging system.

23. A method of using an invasive probe having a transducer assembly, the method comprising:
positioning the invasive probe proximate a region of interest within an anatomical region, wherein the transducer assembly is in a first position and disposed in an outer envelope of the invasive probe;
extending the transducer assembly from within the outer envelope such that the transducer assembly is positioned outside a distal end of the invasive probe;
deploying the transducer assembly to change the position of the transducer assembly from the first position to a second expanded position,
wherein the transducer assembly comprises
a support structure configured to be reversibly changed between a first position and a second position; and
a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.

24. The method of claim 23, further comprising imaging in a first position, a second position, or a position therebetween.

25. The method of claim 23, further comprising energizing the multi-dimensional transducer array in the transducer assembly.

26. The method of claim 23, wherein deploying comprises moving a sliding member along a central guide member of the transducer assembly in a first direction to transition the multi-dimensional transducer array from the first position to the second position.

27. The method of claim 26, further comprising moving the sliding member in a second direction along the central guide member of the transducer assembly to transition the transducer assembly from the second position to the first position, wherein the second direction is opposite to the first direction.

28. The method of claim 27, further comprising retracting the transducer assembly such that the transducer assembly is disposed in the invasive probe.

29. The method of claim 23, further comprising acquiring sensor data via the transducer assembly in the second position.

30. The method of claim 29, further comprising acquiring the sensor data via the transducer assembly to facilitate assessing need for therapy in one or more regions of interest within the anatomical region and delivering therapy to the one or more regions of interest within the anatomical region.

31. The method of claim 30, further comprising employing adaptive signal processing techniques to compensate for variations in the position of each of the plurality of transducer elements to improve the effectiveness of phased array beamforming.

32. The method of claim 30, further comprising generating an image from acquired sensor data for display on a display of a medical imaging system.

33. A method of using an invasive probe having a transducer assembly, the method comprising:
positioning the invasive probe proximate a region of interest within an anatomical region, wherein the transducer assembly is in a first position and disposed in an outer envelope of the invasive probe;
extending the transducer assembly from within the outer envelope such that the transducer assembly is positioned outside a distal end of the invasive probe;
imaging in a first retracted position;
transitioning the position of the transducer assembly from the first retracted position to a second expanded position;
imaging in the second expanded position,
wherein the transducer assembly comprises
a support structure configured to be reversibly changed between a first position and a second position; and
a multi-dimensional transducer array comprising a plurality ‘N’ of sub-groups of transducer elements arranged on the support structure, wherein each of the ‘N’ sub-groups of transducer elements is disposed in a spatial relationship such that an angle formed between each of the ‘N’ sub-groups of transducer elements and at least one other sub-group of transducer elements is less than about 180 degrees, and wherein ‘N’ is an integer.
